(19) United States
(12) Patent Application Publication
(10) Pub. No.: US 2023/0414353 A1
(43) Pub. Date: Dec. 28, 2023

(54) TRANSCATHETER HEART VALVE DELIVERY SYSTEM

(71) Applicant: KINGSTRONBIO (CHANGSHU) CO., LTD., Jiangsu (CN)

(72) Inventors: Shengping ZHONG, Jiangsu (CN); Yongfu JIN, Jiangsu (CN); Chunwang MENG, Jiangsu (CN); Bo ZHANG, Jiangsu (CN)

(21) Appl. No.: 18/463,363

(22) Filed: Sep. 8, 2023

Related U.S. Application Data
(63) Continuation of application No. PCT/CN2021/134832, filed on Dec. 1, 2021.

(30) Foreign Application Priority Data
Mar. 9, 2021 (CN) 202110255628.6

(51) Int. Cl. A61F 2/24

(52) U.S. Cl. CPC A61F 2/2436

(57) ABSTRACT

A transcatheter heart valve delivery system, comprising a valve prosthesis (3), an outer tube (1) and an inner tube (2). The valve prosthesis (3) comprises a valve release end (31), comprising an end face not perpendicular to a valve axis; the outer tube (1) comprises an outer tube release end (11), comprising an end face fitted with the end face of the valve release end (31). An angle between the end face of the outer tube release end and a direction perpendicular to the axis of the outer tube and an angle between the end face of the valve release end and the direction perpendicular to the axis of the outer tube are both within a range of 0 to 60 degrees. The inner tube (2) is telescopically in the outer tube (1) in a penetrating manner. The valve prosthesis (3) is between the inner tube (2) and the outer tube (1).

### TECHNICAL FIELD

The present disclosure relates to the field of cardiac implantable medical device, and in particular, to a transcatheter heart valve delivery system.

### BACKGROUND

Transcatheter Valve Replacement (TVR) is a minimally invasive valve replacement surgery, which refers to implanting an assembled valve by means of a catheter to replace an original valve and functionally achieve valve replacement. The specific implantation process of TVR is as follows: first, an artificial valve is folded in a transcatheter delivery system; then, the valve is delivered to the valve root by using the delivery system through peripheral blood vessels (such as femoral artery, femoral vein, subclavian artery, and active abdominal aorta) or cardiac apex and is then released at the valve root, and is then fixed in the valve annulus. In this way, the original valve is replaced with the artificial valve. TVR has become a main treatment method for patients with valvular stenosis who cannot tolerate surgery or are at high risk for surgery.

At present, an outer tube in the delivery system design generally has a distal end face that is perpendicular to and flush with an axis, because a corresponding valve end face is designed as a flush end face. In recent years, mitral and tricuspid valves implanted through a catheter have appeared, and usually have structures which have irregular release end faces. In this case, if an ordinary delivery system perpendicular to an axis of the outer tube is used for delivery and implantation, beams of the valve stent will jump out successively during the release process of the valve inside the body, and the valve will sway back and forth during the release process, thereby being quite unstable. As a result, the surgeon may not be able to precisely control an implantation position, leading to great difficulties or risks for the surgery. Therefore, it needs to design a matched transcatheter heart valve delivery system.

### SUMMARY

The present disclosure relates to a transcatheter heart valve delivery system, aiming to solve the above-mentioned problem that the delivery system in the prior art cannot match the mitral valve or tricuspid valve.

The purpose of the present disclosure is to provide a transcatheter heart valve delivery system, including: a valve prosthesis, an outer tube, and an inner tube.

The valve prosthesis includes a valve release end, and an end face of the valve release end is not perpendicular to an axis of the valve prosthesis.

The outer tube includes an outer tube release end, and an end face of the outer tube release end is not perpendicular to an axis of the outer tube.

The inner tube is telescopically arranged in the outer tube in a penetrating manner, and a space for accommodating the valve prosthesis is provided between the inner tube and the outer tube. When a proximal end of the valve prosthesis is accommodated in the space, a shape of the end face of the valve release end is consistent with or similar to a shape of the end face of the outer tube release end.

In an embodiment, an angle between the end face of the valve release end and a direction perpendicular to the axis of the outer tube is within a range of 0 to 60 degrees, and an angle between the end face of the outer tube release end and the direction perpendicular to the axis of the outer tube is within a range of 0 to 60 degrees, so as to achieve circumferentially and synchronously release of the valve prosthesis during the release process.

In an embodiment, controllable successive release of the valve prosthesis during the release process can be achieved by adjusting the shape of the outer tube release end.

In an embodiment, the outer tube release end is a plane not perpendicular to the axis of the outer tube or a curved surface.

In an embodiment, the transcatheter heart valve delivery system further includes a tip, the tip is fixedly connected to the end of the inner tube, and a shape of the contact position of the tip and the outer tube is consistent with a shape of the outer tube release end of the outer tube, to form a fitting surface.

In an embodiment, in the accommodated state, a fitting surface of the tip fitted with a distal end of the outer tube is an outer tube release surface, such that this surface of the tip is consistent with the shape of the outer tube release end.

In an embodiment, the diameters of the outer tube and the tip are equal to each other at the fitting position, so as to achieve a smooth transition between the outer surface of the outer tube and the outer surface of the tip.

In an embodiment, the outer tube includes a tube wall having at least one layer.

The technical solution according to the present disclosure can achieve the following beneficial effects.

In the transcatheter heart valve delivery system according to the present disclosure, the shape of the end face of the proximal end of the valve prosthesis can be the same with or similar to the shape of the end face of the distal end of the outer tube, such that the positions of the edge of the end face of the proximal end of the valve prosthesis can be simultaneous separated in the circumferential direction from the release end of the delivery system at the same time, so as to ensure that various parts of the proximal end of the valve prosthesis are synchronously unfolded, ensure the precision of the position of the valve prosthesis inside the human body after being unfolded, and avoid the overall offset of the valve prosthesis resulting from that some parts the release end of the valve prosthesis are first unfolded in the related art. In some embodiments, the shape of the outer tube release end can also be designed according to the shape of the valve release end, so as to realize the specific requirements for the successive release of each beam of the stent of the valve. In addition, the end face of the outer tube release end is not perpendicular to the axis of the outer tube, such that images of different shapes can be obtained in a lateral direction of the outer tube when the outer tube rotates, and thus an angle and the position that the outer tube rotates can be directly determined and can be used as angle positioning marks during the implantation process.

It should be understood that both the foregoing general description and the following detailed description are just exemplary, but not restrictive to the application.

### BRIEF DESCRIPTION OF DRAWINGS

FIG. 1 is a schematic structural diagram of a transcatheter heart valve delivery system according to an embodiment of the present disclosure.

FIG. 2 is a partial enlarged view of a transcatheter heart valve delivery system according to an embodiment of the present disclosure.

FIG. 3 is a diagram showing an unfolded state of the valve prosthesis.

### REFERENCE NUMERALS

1—outer tube;
11—outer tube release end;
2—inner tube;
3—valve prosthesis;
31—valve release end;
4—tip; and
5—handle assembly.

The accompanying drawings herein are incorporated into, and constitute a part of, this specification, illustrate embodiments consistent with the present disclosure, and serve to explain the principles of the present disclosure together with this specification.

### DESCRIPTION OF EMBODIMENTS

In order to better illustrate the purpose, technical solutions and advantages of the present disclosure, the present disclosure will be further described in detail below with reference to the drawings and embodiments. It should be understood that the specific embodiments described herein are used just to explain the present disclosure, but not to limit the present disclosure.

In the description of the present disclosure, unless otherwise expressly stipulated and defined, the terms such as “first” and “second” are merely used for descriptive purposes, but shall not be illustrated as indicating or implying relative importance. Unless otherwise specified or stated, the term “a plurality of” or “multiple” refers to two or more; the terms such as “connection”, “fixed/fixation”, etc. shall be illustrated in a broad sense. For example, “connection” may refer to “fixedly connection”, “detachably connection”, “integrally connection”, or “electrically connection”; and/or directly connection or indirectly connection through an intermediary. For one skilled in the art, the specific meanings of the above terms in the present disclosure can be understood according to specific circumstances.

In the description of this specification, it should be understood that the directional words such as “above/upper” and “below/lower” described in embodiments of the present disclosure are described from the perspective shown in the drawings, but cannot be illustrated as a limitation to the embodiments of the present disclosure. In addition, it should be understood in the context that an element is connected “above” or “below” another element, the element may be directly connected “above” or “below” the another element, or the element may be indirectly connected “above” or “below” the another element through an intermediate element.

A valve is an important organ in the process of human blood circulation, and is configured to be passively open or closed to ensure that the blood circulates in one direction. The valve needs to be treated in time when there is something wrong with it, otherwise it is prone to be life-threatening. A Patient undergoes valve replacement surgery by using an artificial valve to replace an original valve. Generally, valve replacement surgery is performed inside the heart, and thus requires for high precision in the surgery, especially for precision of a position of the valve release.

Valve replacement surgery usually requires for a special delivery system used for delivering a valve prosthesis. First, the valve prosthesis is folded and accommodated in the delivery system, and is delivered by the delivery system to a preset position in a human body; then, the valve prosthesis is gradually released by a control mechanism at the release end of the delivery system, such that the valve prosthesis can be released out from the release end of the delivery system. When the valve release end of the valve prosthesis is separated from the release end of the delivery system, the valve prosthesis is completely released, and is completely unfolded automatically or through balloon expansion at the preset position, thereby achieving replacement of the original valve inside the human body.

However, it should be noted that in the related art, the end face at the exit of the release end of the delivery system is perpendicular to the axis of the outer tube of the delivery system, and the delivery system with such a structure is merely applicable to the valve prosthesis of which the end face of the valve release end is perpendicular to the axis. That is, at the moment when the valve release end of the valve prosthesis is separated from the release end of the delivery system, an edge of the valve release end of the valve prosthesis can be separated from the release end of the delivery system at the same time, to ensure the precision of the position of the valve prosthesis during the expansion process.

However, for interventional valves such as a mitral valve and a tricuspid valve, the release end of this type of valve prosthesis 3 is usually designed to have an end face not perpendicular to the axis, and the end face of the release end of the corresponding delivery system should match the valve release end 31. When this type of valve prosthesis 3 is separated from the release end of the delivery system in the related art, because the end face of the release end of the delivery system in the related art is a plane, some parts of the edge of the valve release end 31 of the valve prosthesis 3 are first separated from the release end of the delivery system while some other parts of the edge of the valve release end 31 of valve prosthesis 3 are not separated from the outlet of the delivery system yet. In this case, the edge of the valve release end 31 of valve prosthesis 3 that is first separated from the delivery system is first automatically unfolded, and the edge that is not separated from the delivery system yet is still restricted by the delivery system and thus cannot be unfolded. As a result, the valve release end 31 of the valve prosthesis 3 cannot be unfolded at the same time when separated from the delivery system, thereby leading to offset of the valve prosthesis 3 to a side which is first unfolded, and thus making it difficult to ensure the precision of the position of valve prosthesis 3 inside the human body, and bringing a safety hazard to human health.

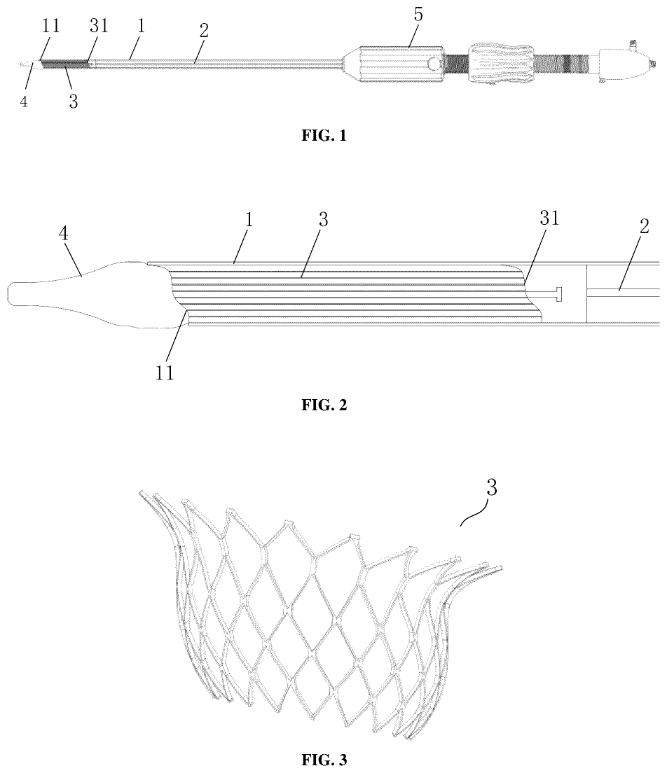

In view of this, as shown in FIG. 1 to FIG. 3, the present disclosure provides a transcatheter heart valve delivery system, including an outer tube 1, an inner tube 2 and a valve prosthesis 3. The valve prosthesis 3 includes a valve release end 31, and the end face of the valve release end 31 is not perpendicular to the axis of valve prosthesis 3. The outer tube 1 includes an outer tube release end 11. The end face of the outer tube release end 11 is not perpendicular to the axis of the outer tube 1. When in use, the delivery system can be operated in a hand-held manner by a medical staff. The outer tube release end 11 is an end away from the medical staff.

In addition, the inner tube 2 is telescopically arranged in the outer tube 1 in a penetrating manner, and a space for accommodating the valve prosthesis 3 is provided between the inner tube 2 and the outer tube 1. The inner tube 2 and the outer tube 1 are arranged coaxially, and the space for accommodating the valve prosthesis 3 is formed between an outer wall of inner tube 2 and an inner wall of the outer tube 1.

When valve release end 31 of valve prosthesis 3 is accommodated in the above-mentioned space, the shape of the end face of valve release end 31 of valve prosthesis 3 is consistent with or similar to the shape of the end face of outer tube release end 11 of outer tube 1. The valve release end 31 of the valve prosthesis 3 refers to the end adjacent to the medical staff. The valve prosthesis 3 can be made of memory metal, and an overall volume thereof may become smaller after being folded, such that it can be accommodated in the space formed between the outer tube 1 and inner tube 2. In this case, the shape of the end face of the valve release end 31 of the valve prosthesis 3 is the same as the shape of the end face shape of outer tube release end 11 of outer tube 1.

In this embodiment, when the valve release end 31 of the valve prosthesis 3 with the end face not perpendicular to the axis is separated from the outlet of the outer tube release end 11 of the delivery system, since the shape of the end face of the valve release end 31 of the valve prosthesis 3 is the same as the shape of the end face of the outer tube release end 11 of the outer tube 1, the edge of the end face of the valve release end 31 of the valve prosthesis 3 at different positions can be separated from the outer tube release end 11 of the delivery system at the same time. In this way, all parts of the release end 31 of the valve prosthesis 3 can be synchronously unfolded, thereby ensuring the precision of the position of valve prosthesis 3 inside the human body after being unfolded, and thus avoiding the overall offset of the valve prosthesis 3 in the related art resulting from that parts of valve release end 31 of valve prosthesis 3 are first unfolded. It can be understood that, in order to enable the transcatheter heart valve delivery system to match valve prosthesis 3 with different shapes of end faces so as to achieve the purpose of releasing the valve prosthesis 3 in an order required by the design, the shape of the end face of the outer tube release end 11 can be designed according to the structure and the shape of the valve release end 31 of the valve prosthesis 3 to be delivered.

In addition, it should be noted that, during the process that the valve prosthesis 3 is delivered to the preset position inside the human body through the delivery system, if an angle at which the valve prosthesis 3 is unfolded deviates, the unfolding angle of the valve prosthesis 3 needs to be adjusted by rotating the delivery system. However, in the related art, the end face of the outer tube release end 11 of the outer tube 1 is a plane perpendicular to the axis of the outer tube 1, and it is difficult to identify the rotation angle of the outer tube 1 in a lateral direction of the outer tube 1. In the related art, end face ultrasound technology or X-ray scanning technology is generally used to assist in determining the angle of valve prosthesis 3. In order to facilitate identifying the angle of the outer tube 1 by ultrasound or X-rays, a development point is generally provided on the valve prosthesis 3. The development point may be made of metal. When the ultrasound or the X-rays irradiate on the development point, an angle of the valve prosthesis 3 can be determined according to the position of the development point. The metal material of the developing point is usually a noble metal material.

In the present disclosure, the end face of the outer tube release end 11 of the outer tube 1 is a plane that is not perpendicular to the axis of the outer tube 1 or a curved surface, such as an inclined plane, a special-shaped surface. Therefore, when the outer tube 1 rotates, images of different shapes can be obtained in the lateral direction of the outer tube 1, such that the angle and the position that the outer tube 1 rotates can be directly determined without providing a development point on outer tube 1, thereby facilitating the operation and forming process of the outer tube 1.

In an embodiment, an angle between the end face of the valve release end 31 and the direction perpendicular to the axis of outer tube 1 is within a range of 0 to 60 degrees, and an angle between the end face of outer tube release end 11 and the direction perpendicular to the axis of outer tube 1 is within a range of 0 to 60 degrees. Within this range, the end face of valve release end 31 can effectively match the end face of the outer tube release end 11.

It can be understood that, in order to facilitate the transcatheter heart valve delivery system to enter the human vein and to move inside the human body, the tip 4 can be fixedly connected to the end of the inner tube 2, such that the tip 4 can provide orientation for the movement of the transcatheter heart valve delivery system.

It should be noted that the fitting surfaces of the tip 4 and the outer tube 1 should be in as close contact and as consistent with each other as possible, such that during the movement process of the delivery system, the human tissue damage or movement impediment due to the seam formed by the mismatch between the tip 4 and the outer tube 1 can be prevented. For this reason, the contact surface of the tip 4 and the outer tube 1 should also be designed as an end face structure consistent with the outer tube release end 11.

In an embodiment, as shown in FIG. 1 and FIG. 2, the end face of the tip 4 is also a surface not perpendicular to the axis, and the tip 4 is in close contact with the end face of the outer tube release end 11 of the outer tube 1. Before the valve prosthesis 3 is delivered to the preset position inside the human body, the end face of the tip 4 can be closely fitted with the end face of the outer tube release end 11, so as to seal the valve prosthesis 3 in the space between the outer tube 1 and the inner tube 2. When the delivery system reaches the preset position inside the human body, the inner tube 2 can be controlled to move relative to the outer tube 1, such that the tip 4 moves together with the inner tube 2 towards a direction away from the outer tube release end 11. During this process, the valve prosthesis 3 moves synchronously with the inner tube 2, to gradually extend out of the outer tube release end 11. When the valve release end 31 of the valve prosthesis 3 is completely separated from the outer tube release end 11, the valve prosthesis 3 can be completely unfolded and separated from the inner tube 2 to achieve replacement operation of the valve.

In an embodiment, in order to make the fitting position of the tip 4 and the outer tube release end 11 seamless, the shape of the end face of the tip 4 is consistent with the shape of the end face of the outer tube release end 11.

Further, the diameters of the outer tube 1 and the tip 4 are equal to each other at the fitting position, so as to achieve a smooth transition between the outer surface of the outer tube 1 and the outer surface of the tip 4. That is, when the end face of the tip 4 is fitted with the end face of the outer tube release end 11, the side wall of the tip 4 and the side wall of the outer tube 1 can form a side wall with a smooth transition, which is conductive to moving inside the human body, thereby avoiding damage to the human body.

In addition, the outer tube 1 may include a tube wall having at least one layer. That is, the tube wall of the outer tube 1 may have only one layer, two layer, or three or more layers. In this embodiment, the tube wall of outer tube 1 has only one layer.

It can be understood that in some designs the beams of the stent of the release end of the valve may need to be successively released. Properly designing the structure of the outer tube release end 11 according to the present disclosure can realize the pre-designed release order.

It can be understood that, in order to facilitate controlling the movement of the inner tube 2 inside the outer tube 1, the transcatheter heart valve delivery system may further include a handle assembly 5 connected to the inner tube 2, and the medical staff can control the movement of the inner tube 2 and the overall movement of the transcatheter heart valve delivery system by hand-holding the handle assembly 5.

The above description merely describes some preferred embodiments of the present disclosure, and shall not be illustrated as to limit the present disclosure. For one skilled in the art, various changes and variations can be made to the present disclosure. Any modifications, equivalent replacements, improvements or the like made without departing from the principle and essence of the present disclosure shall fall within the protection scope of the present disclosure.

What is claimed is:

1. A transcatheter heart valve delivery system, comprising:
a valve prosthesis (3), wherein the valve prosthesis (3) comprises a valve release end (31), and an end face of the valve release end (31) is not perpendicular to an axis of the valve prosthesis (3);
an outer tube (1), wherein the outer tube (1) comprises an outer tube release end (11), and an end face of the outer tube release end (11) is not perpendicular to an axis of the outer tube (1);
an inner tube (2), wherein the inner tube (2) telescopically arranged in the outer tube (1) in a penetrating manner, and a space for accommodating the valve prosthesis (3) is provided between the inner tube (2) and the outer tube (1), and wherein when the valve prosthesis (3) is accommodated in the space, a shape of the end face of the valve release end (31) of the valve prosthesis (3) is consistent with or similar to a shape of the end face of the outer tube release end (11) of the outer tube (1).

2. The transcatheter heart valve delivery system according to claim 1, wherein an angle between the end face of the valve release end (31) and a direction perpendicular to the axis of the outer tube is within a range of 0 to 60 degrees, and an angle between the end face of the outer tube release end (11) and the direction perpendicular to the axis of the outer tube is within a range of 0 to 60 degrees.

3. The transcatheter heart valve delivery system according to claim 1, wherein the end face of the outer tube release end (11) is a plane or a curved surface not perpendicular to the axis of the outer tube (1).

4. The transcatheter heart valve delivery system according to claim 2, wherein the end face of the outer tube release end (11) is a plane or a curved surface not perpendicular to the axis of the outer tube (1).

5. The transcatheter heart valve delivery system according to claim 1, further comprising a tip (4), wherein the tip (4) is fixedly connected to an end of the inner tube (2), and a shape of a contact position between the tip (4) and the outer tube (1) is consistent with a shape of the outer tube release end (11) of the outer tube (1) to form a fitting surface.

6. The transcatheter heart valve delivery system according to claim 1, wherein the outer tube (1) comprises a tube wall having at least one layer.
